(19) United States
(12) Patent Application Publication
(10) Pub. No.: US 2019/0240078 A1
(43) Pub. Date: Aug. 8, 2019

(54) Smart diaper capable of sensing the size of a soiled area

(71) Applicant: Gallop Creation USA Inc., Milpitas, CA (US)

(72) Inventors: Li Li, Femont, CA (US); Zhigang Wang, Fremont, CA (US)

(21) Appl. No.: 15/889,375

(22) Filed: Feb. 6, 2018

(51) Int. Cl. A61F 13/42; G01N 27/04; G01B 7/02

(52) U.S. Cl. CPC A61F 13/42; G01N 27/048; A61F 2013/423; A61F 2013/424; G01B 7/02

(57) ABSTRACT

A smart diaper to be worn by a user, includes an absorption material configured to absorb to form a soiled area, a pair of electrode sensing circuits comprising a first electrode sensing circuit and a second electrode sensing circuit, which are positioned parallel to each other and are in contact with the absorbent material, wherein the pair of electrode sensing circuits can be laid out in a spatially distributed pattern across the absorption material, and a controller that can apply a voltage across the pair of electrode sensing circuits and to produce an electrical current that increases with area of the soiled area according to the spatially distributed pattern.

### BACKGROUND OF THE INVENTION

The present application relates to a smart diaper that can sense whether a diaper has been soiled, and in particular, to a diaper which can sense the size and the type of soiling.

There has been a long-felt need for the detection of soiled diapers and for caregivers to receive real-time notifications about soiled diapers so that they can be promptly changed. Diaper soil detection and alert are important for babies, who could develop diaper rash when left wearing soiled diapers for long periods of time, as well as for incontinent adults who are unable to communicate with attendants and are susceptible to infections caused by wet diapers.

For cost reasons, many smart diaper products rely on reusable sensors. The caregiver needs to remove and to reattach a reusable sensor during each diaper change. If a disposable sensor is embedded into the diaper, the diaper change process will be further simplified, and a better user experience achieved. However, the cost of a single-use smart diaper must be sufficiently low to be accepted by consumers.

### SUMMARY OF THE INVENTION

The presently application discloses a smart diaper that can sense the size or the wet area in diaper. The disclosed smart diaper is reusable and is embedded with a conductivity sensor. The sensor can be connected to a controller that can detect wetting of the diaper from measured signals, computing wet area, and transmitting a (wireless) signal to an external device such as a smart phone to alert caretakers. The controller can also send signal to a server, which can send a message to alert a caregiver. The measurements can be performed without affecting users' normal daily activities.

Moreover, the sensor in the disclosed smart diaper can be spatially distributed to facilitate sensing the degree or the area, and the location of the wetting. In particular, the sensor can be implemented as a sensing circuit that is laid out in a spread area of the diaper.

Furthermore, the disclosed smart diaper overcomes the problem of false alarm in conventional technologies. Because the disclosed diaper can detect the size of the wet area in diaper, alert signals are sent only when the sizes of the wet area exceed a threshold. Alternatively, alert signals can indicate the size of the wet area to give caretaker a sense of urgency for diaper change.

In one general aspect, the present invention relates to a smart diaper to be worn by a user, that includes an absorption material that can absorb to form a soiled area; a pair of electrode sensing circuits comprising a first electrode sensing circuit and a second electrode sensing circuit, which are positioned parallel to each other and are in contact with the absorbent material, wherein the pair of electrode sensing circuits is laid out in a spatially distributed pattern across the absorption material; and a controller that can apply a voltage across the pair of electrode sensing circuits and to produce an electrical current that increases with area of the soiled area according to the spatially distributed pattern.

Implementations of the system may include one or more of the following. The pair of electrode sensing circuits can include multiple curved portions, wherein the distance between adjacent curved portions can be at least 5 times larger than a gap between the first electrode sensing circuit and the second electrode sensing circuit. The distance between adjacent curved portions can be at least 10 times larger than a gap between the first electrode sensing circuit and the second electrode sensing circuit. The pair of electrode sensing circuits can include multiple straight portions, wherein the distance between adjacent straight portions can be at least 5 times larger than a gap between the first electrode sensing circuit and the second electrode sensing circuit. The distance between adjacent straight portions can be at least 10 times larger than a gap between the first electrode sensing circuit and the second electrode sensing circuit. The pair of electrode sensing circuits can be laid out in a serpentine shape, a circular shape, a spiral shape, or an oval shape. The controller can produce a current that is substantially proportional to the area of the soiled area according to the spatially distributed pattern. The absorption material can form an absorption layer, wherein the first electrode sensing circuit is positioned above the second electrode sensing circuit relative to the absorption layer. The absorption material forms an absorption layer, wherein the first electrode sensing circuit and the second electrode sensing circuit relative to the absorption layer are positioned side by side relative to the absorption layer. The pair of electrode sensing circuits is embedded in the absorbent material. The controller can include a semiconductor chip in connection with the pair of electrode sensing circuits and configured to produce the voltage and to measure the electrical current that increases with the area of the soiled area. The controller can include an antenna configured to transmit a wireless signal to alert a caregiver based on the electrical current measured by the semiconductor chip. The wireless signal can alert the caregiver when the electrical current measured the controller passes a threshold value. The wireless signal can indicate area of the soiled area.

In another aspect, the present invention relates to a smart diaper to be worn by a user, that includes a segmented measurement strip comprising: a first electrode sensing circuit and a second electrode sensing circuit, which are positioned parallel to each other, a plurality segments of absorbent material between the first electrode sensing circuit and the second electrode sensing circuit, wherein the absorption material can absorb a liquid; and segments of non-absorbent material between the plurality segments of absorbent material; and a controller that can apply a voltage across the first electrode sensing circuit and the second electrode sensing circuit and to produce an electrical current that increases with number of segments of absorbent material that is soiled by the liquid.

Implementations of the system may include one or more of the following. The smart diaper can further include an absorption layer configured to absorb the liquid to form a soiled area, wherein the absorption layer can be in contact with the plurality segments of absorption material. The non-absorbent material can include a hydrophobic film to enhance liquid resistance. The first electrode sensing circuit and the second electrode sensing circuit can be formed by a conductive ink placed on a layer of the absorbent material, wherein the segments of non-absorbent material can be formed by coating a hydrophobic film on the layer of absorbent material. The controller can include a semiconductor chip in connection with the pair of electrode sensing circuits and configured to produce the voltage and to measure the electrical current that increases with the area of the soiled area. The controller can include an antenna configured to transmit a wireless signal to alert a caregiver based on the electrical current measured by the semiconductor chip.

These and other aspects, their implementations and other features are described in detail in the drawings, the description and the claims.

### BRIEF DESCRIPTION OF THE DRAWINGS

FIG. 1 illustrates a smart diaper in accordance with some embodiments of the present application.

FIG. 2A is a top view of a portion of a smart diaper having a pair of electrode sensing circuits in accordance with some embodiments of the present application.

FIG. 2B is a cross-sectional view of the smart diaper having a pair of electrode sensing circuits along the direction A-A in FIG. 2A.

FIG. 3A is a top view of a portion of another smart diaper having a pair of electrode sensing circuits in accordance with some embodiments of the present application.

FIG. 3B is a cross-sectional view of the smart diaper having a pair of electrode sensing circuits along the direction B-B in FIG. 3A.

FIG. 4 is a top view of a portion of another smart diaper having a segmented electrode assembly in accordance with some embodiments of the present application.

### DETAILED DESCRIPTION OF THE INVENTION

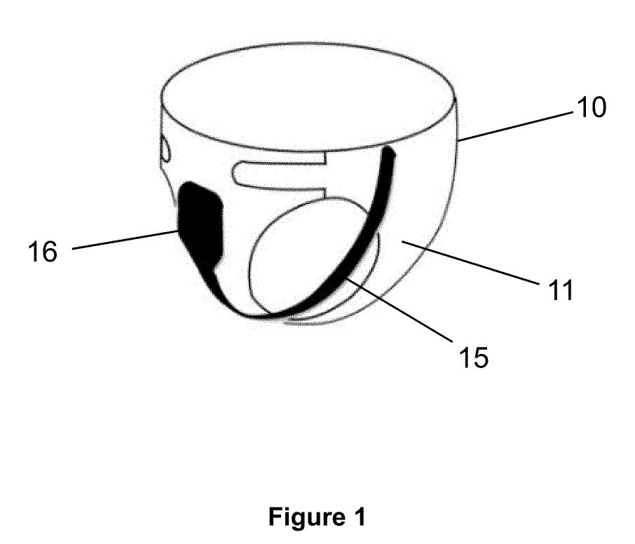

Referring to FIG. 1, a smart diaper 10 includes an absorption material 11, sensing electrodes 15, and a controller 16. The controller 16 also includes a semiconductor chip configured to process measurement signals, a battery configured to power the sensing electrodes 15 and the semiconductor chip, an antenna configured to wirelessly transfer data to an external device, and other electronic components. For example, the semiconductor chip can produce a voltage to be applied to sensing electrodes 15 and measure an electrical current across the sensing electrodes 15. The wirelessly transferred data can include wetting status, and/or the degree of wetting or the size of the wetting area in the smart diaper 10.

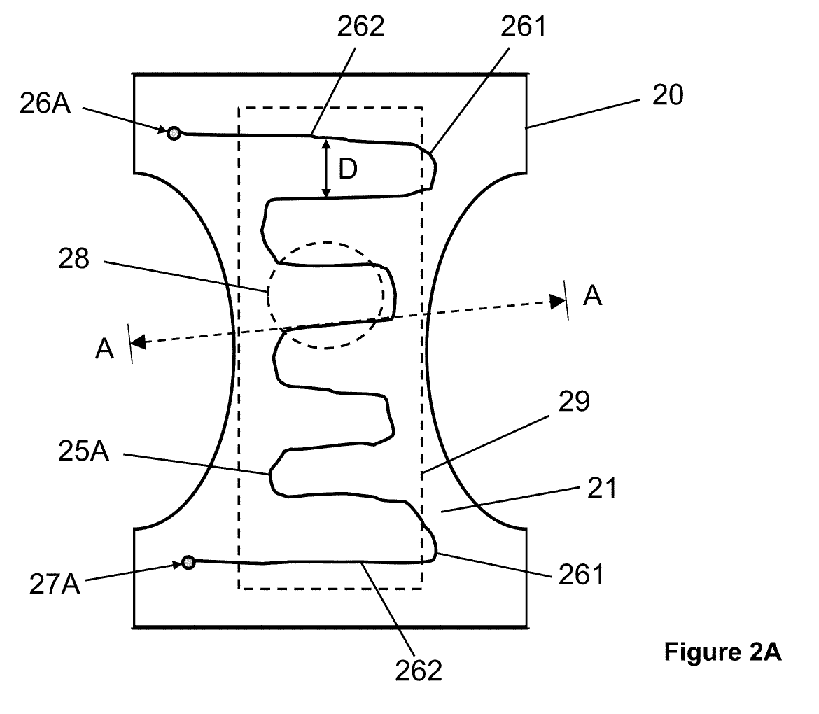

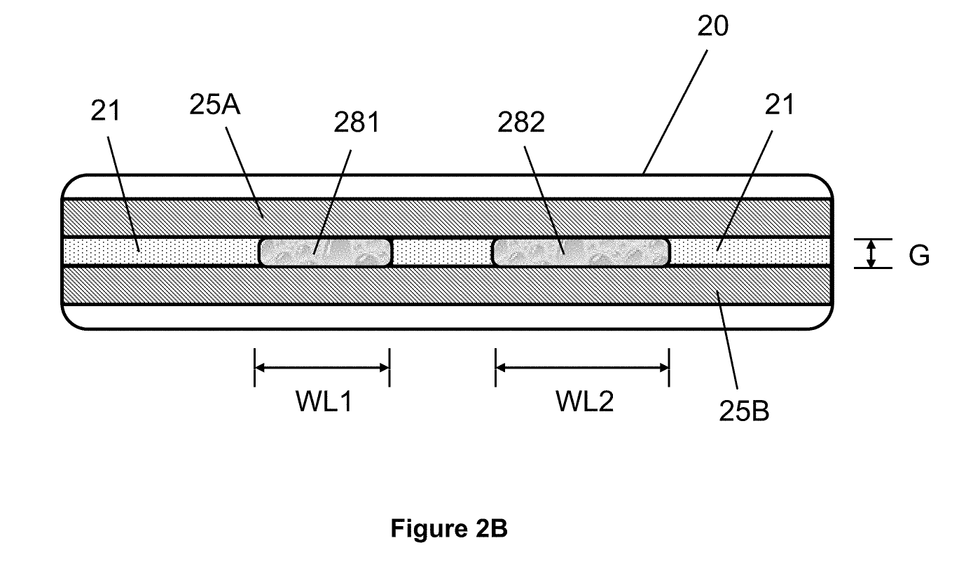

In some embodiments, FIG. 2A shows a top view of a portion of a smart diaper 20, and FIG. 2B shows a cross-sectional view of the smart diaper 20 along the direction A-A in FIG. 2A. The smart diaper 20 includes an absorbent material 21 and a pair of electrode sensing circuits: an upper electrode sensing circuit 25A and a lower electrode sensing circuit 25B, which are positioned parallel to each other and are in contact with or embedded in the absorbent material 21. The absorption material 21 can form an absorption layer, wherein the upper electrode sensing circuit 25A is positioned above the lower electrode sensing circuit 25B relative to the absorption layer.

The upper electrode sensing circuit 25A can each include connection pads 26A, 27A configured to connect the upper electrode sensing circuit 25A to a controller (e.g. 16 in FIG. 1). Similarly, the lower electrode sensing circuit 25B can also include connection pads (not shown in FIG. 2A) for connecting it to the controller. The controller (e.g. 16 in FIG. 1) can apply an electric voltage across the upper electrode sensing circuit 25A and the lower electrode sensing circuit 25B, and measures the electric current running through the pair of electrode sensing circuits 25A, 25B. The voltage can be applied and current measurements conducted periodically, for example at 20-second intervals, to save power.

One advantageous feature of the presently disclosed smart diaper is that the pair of electrode sensing circuits 25A, 25B are laid out in a spatially distributed pattern across the absorption material 21 to cover the most commonly soiled area 29 when the smart diaper is wet. In some embodiments, the upper electrode sensing circuit 25A and the lower electrode sensing circuit 25B include multiple curved portions 261 and multiple substantially straight portions 262 to allow each electrode sensing circuit to cover the most commonly soiled area 29. In some embodiments, the upper electrode sensing circuit 25A and the lower electrode sensing circuit 25B are laid out in a serpentine shape, or a circular, spiral, or oval shape to cover the most commonly soiled area 29.

In some embodiments, the upper electrode sensing circuit 25A and the lower electrode sensing circuit 25B are printed using a conductive ink on a portion of the absorbent material 21 to cover the most commonly soiled area 29, which are subsequently embedded by another portion the absorbent material 21.

When the diaper 20 is wet, a wet area 28 is formed in the absorbent material 21. The wet area 28 may include one or more soiled portions 281, 282 between the upper electrode sensing circuit 25A and the lower electrode sensing circuit 25B. The dry absorbent material 21 has a very high resistivity (and low electrical conductivity), while the soiled dry absorbent material 21 has a much lower resistivity (and high electrical conductivity). When a voltage is applied across the upper electrode sensing circuit 25A and the lower electrode sensing circuit 25B by the controller (16 in FIG. 1), the current running between the upper electrode sensing circuit 25A and the lower electrode sensing circuit 25B increases, which indicates wetting of the absorbent material 21.

An important feature of the presently disclosed smart diapers is that the current strength dependent on the total length of the soiled portions between the upper electrode sensing circuit 25A and the lower electrode sensing circuit 25B. For example, in FIG. 2B, the total length of the soiled portions 281, 282 is WL1+WL2. In the disclosed smart diaper, the gap G between the upper electrode sensing circuit 25A and the lower electrode sensing circuit 25B is much smaller than the average distance D between adjacent curved portions 261 or adjacent straight portions 262, which allows measured currents approximately proportional to the total length of the soiled portions between the pair of electrode sensing circuits. For example, D>=10G, or D>=5G. Thus, there is effectively a parallel electrical circuit between the upper electrode sensing circuit 25A and the lower electrode sensing circuit 25B. The total current running in the parallel electrical circuit is the sum of currents through different portions along the length of the upper electrode sensing circuit 25A and the lower electrode sensing circuit 25B. Thus, the current increases with (approximately proportional to considering the wet conductivity is much larger than the dry conductivity of the absorbent material 21) the total length of the soiled portions between the upper electrode sensing circuit 25A and the lower electrode sensing circuit 25B.

Another important feature of the presently disclosed smart diapers is that the current strength increases with, and approximately proportional to, the total area of the wet area(s) 28. Since the upper electrode sensing circuit 25A and the lower electrode sensing circuit 25B are laid out in a spatially distributed pattern to cover the most commonly soiled area 29 (e.g. as shown in FIG. 2A), the total area of the wet area(s) 28 is approximately proportional to the total length of the soiled portions between the upper electrode sensing circuit 25A and the lower electrode sensing circuit 25B.

Thus, the current strength measured by the controller (16 in FIG. 1) reflects the total soiled area in the disclosed smart diaper. In some embodiments, the measured current strength is processed to calculate a soiled area in the smart diaper (10, 20). An alert notification can be sent to an external device if the soiled area calculated is larger than a threshold. Alternatively, the soiled area or degree of soiling of the smart diaper is sent to an external device. Upon received the notification, the caregiver can promptly change the smart diaper for the user.

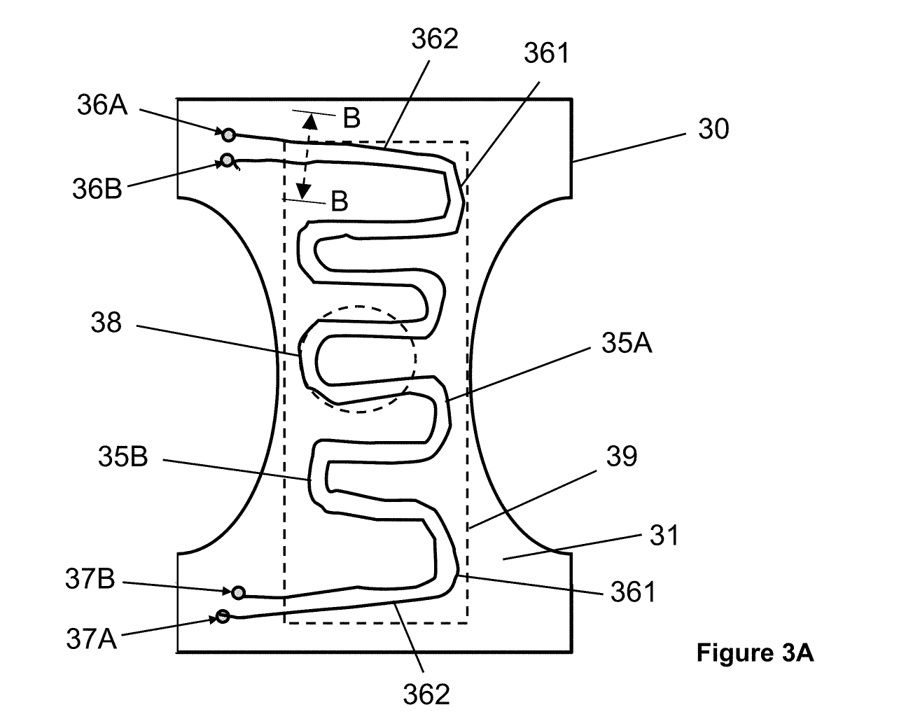

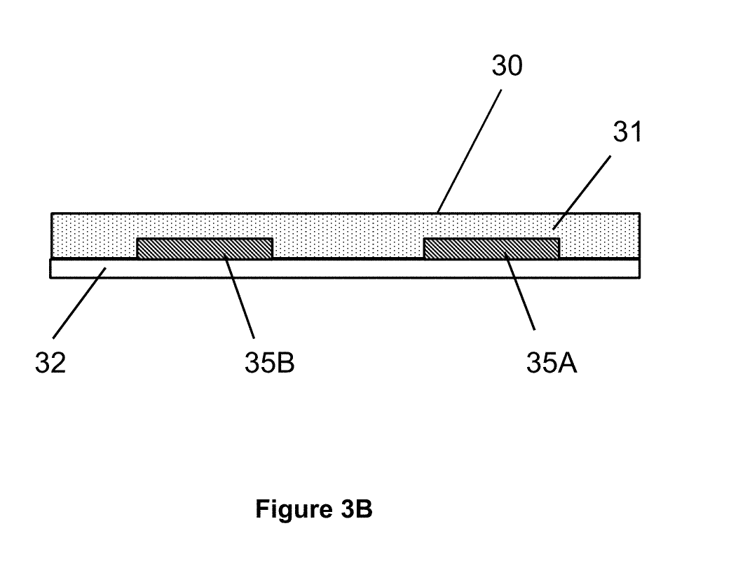

In some embodiments, a pair of electrode sensing circuits can also be laid out side by side on the plane of the absorbent material in a smart diaper. Referring to FIGS. 3A and 3B, a smart diaper 30 includes an absorbent material 31, and a pair of electrode sensing circuits: a first electrode sensing circuit 35A and a second electrode sensing circuit 35B, which are laid out side by side on (in the planar direction of) a substrate layer 32 and inside the absorbent material 31. The first electrode sensing circuit 35A and the second electrode sensing circuit 35B are positioned parallel to each other and are in contact with or embedded in the absorbent material 21. The absorption material 31 can form an absorption layer, the first electrode sensing circuit 35A and the second electrode sensing circuit 35B are positioned side by side relative to the absorption layer. The absorbent material 31 can absorb liquid and the substrate layer 32 is made of a water resistant material.

The first electrode sensing circuit 35A can each include connection pads 36A, 37A configured to connect the first electrode sensing circuit 35A to a controller (e.g. 16 in FIG. 1). Similarly, the second electrode sensing circuit 35B can also include connection pads 36B, 37B for connecting it to the controller. The controller (e.g. 16 in FIG. 1) can apply an electric voltage across the first electrode sensing circuit 35A and the second electrode sensing circuit 35B, and measures the electric current running through the pair of electrode sensing circuits 35A, 35B. The voltage can be applied and current measurements conducted periodically, for example at 20-second intervals, to save power.

One advantageous feature of the presently disclosed smart diaper is that the pair of electrode sensing circuits 35A, 35B are laid out in a spatially distributed pattern across the absorption material 31 to cover the most commonly soiled area 39 when the smart diaper is wet. In some embodiments, the first electrode sensing circuit 35A and the second electrode sensing circuit 35B include multiple curved portions 361 and multiple substantially straight portions 362 to allow each electrode sensing circuit to cover the most commonly soiled area 39. In some embodiments, the first electrode sensing circuit 35A and the second electrode sensing circuit 35B are laid out in a serpentine shape or a circular, spiral, or oval shape to cover the most commonly soiled area 39.

In some embodiments, the first electrode sensing circuit 35A and the second electrode sensing circuit 35B are printed using a conductive ink on a portion of the absorbent material 31 to cover the most commonly soiled area 39, which are subsequently embedded by another portion the absorbent material 31.

When the diaper 30 is wet, a wet area 38 is formed in the absorbent material 31. The wet area 38 may include one or more soiled portions between the first electrode sensing circuit 35A and the second electrode sensing circuit 35B. The dry absorbent material 31 has a very high resistivity (and low electrical conductivity), while the soiled dry absorbent material 31 has a much lower resistivity (and high electrical conductivity). When a voltage is applied across the first electrode sensing circuit 35A and the second electrode sensing circuit 35B by the controller (16 in FIG. 1), the current running between the first electrode sensing circuit 35A and the second electrode sensing circuit 35B increases, which indicates wetting of the absorbent material 31.

An important feature of the presently disclosed smart diapers is that the current strength dependent on the total length of the soiled portions between the first electrode sensing circuit 35A and the second electrode sensing circuit 35B. In the disclosed smart diaper, the gap between the first electrode sensing circuit 35A and the second electrode sensing circuit 35B is much smaller than the average distance between adjacent curved portions 361 or adjacent straight portions 362, for example, having a ratio 1:10 or 1:5. Thus, there is effectively a parallel electrical circuit between the first electrode sensing circuit 35A and the second electrode sensing circuit 35B. The total current running in the parallel electrical circuit is the sum of currents through different portions along the length of the first electrode sensing circuit 35A and the second electrode sensing circuit 35B. Thus, the current increases with (approximately proportional to considering the wet conductivity is much larger than the dry conductivity of the absorbent material 31) the total length of the soiled portions between the first electrode sensing circuit 35A and the second electrode sensing circuit 35B.

Another important feature of the presently disclosed smart diapers is that the current strength dependent on, and approximately proportional to, the total area of the wet area(s) 38. Since the first electrode sensing circuit 35A and the second electrode sensing circuit 35B are laid out in a spatially distributed pattern to cover the most commonly soiled area 39 (e.g. as shown in FIG. 3A), the total area of the wet area(s) 38 is approximately proportional to the total length of the soiled portions between the first electrode sensing circuit 35A and the second electrode sensing circuit 35B.

Thus, the current strength measured by the controller (16 in FIG. 1) reflects the total soiled area in the disclosed smart diaper. In some embodiments, the measured current strength is processed to calculate a soiled area in the smart diaper (10, 20, 30). An alert notification can be sent to an external device if the soiled area calculated is larger than a threshold. Alternatively, the soiled area or degree of soiling of the smart diaper is sent to an external device. Upon received the notification, the caregiver can promptly change the smart diaper for the user.

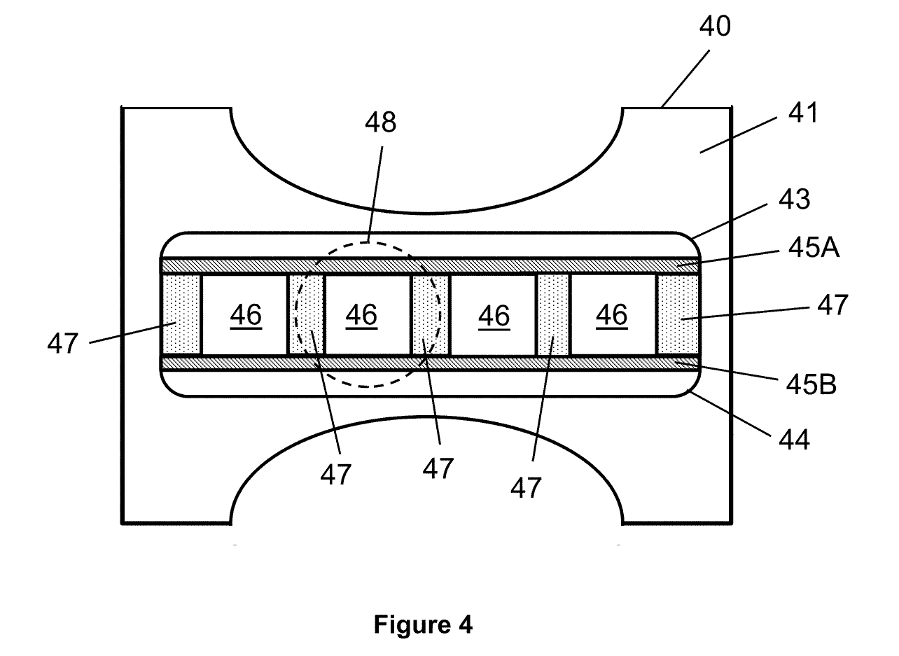

In some embodiments, referring to FIG. 4, a smart diaper 40 includes an absorbent layer 41 and a segmented measurement strip 43 on or in the absorbent layer 41. The segmented measurement strip 43 includes a pair of electrode sensing circuits: a first electrode sensing circuit 45A and a second electrode sensing circuit 45B that are in parallel with each other. The gap between the first electrode sensing circuit 45A and the second electrode sensing circuit 45B is filled with a plurality segments of absorbent material 46, which are separated by segments of a non-absorbent material 47 in contact with the absorption layer. The non-absorbent material 47 can be made by coating an absorbent material with a hydrophobic film to enhance liquid resistance. For example, the hydrophobic film can include self-assembled monolayers of Perfluorodecyltrichlorosilane (FDTS).

In some embodiments, the first electrode sensing circuit 45A and the second electrode sensing circuit 45B can be constructed by delivering a conductive ink film on a uniform layer of the absorbent material 46 in the spatial pattern of the two electrode sensing circuits. The segments of the non-absorbent material 47 can be made by coating a hydrophobic film on the uniform layer of the absorbent material 46 in a spatial pattern defined by the segments of the non-absorbent material 47.

When a wet area 38 is formed during usage, one or more segments of the absorbent material 46 (possibly also portions in the absorbent layer 41) are soiled. The current measured by the controller (16 in FIG. 1) increases. The current increase is approximately proportional to the number of soiled segments of the absorbent material 46. An alert notification can be sent to an external device if at least one segment of the absorbent material 46 is soiled. The number of segments of the absorbent material 46 soiled areas can also be sent to an external device. Upon received the notification, the caregiver can promptly change the smart diaper for the user.

While this document contains many specifics, these should not be construed as limitations on the scope of an invention that is claimed or of what may be claimed, but rather as descriptions of features specific to particular embodiments. Certain features that are described in this document in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination.

Only a few examples and implementations are described. Other implementations, variations, modifications and enhancements to the described examples and implementations may be made without deviating from the spirit of the present invention.

What is claimed is:

1. A smart diaper to be worn by a user, comprising:
an absorption material configured to absorb to form a soiled area;
a pair of electrode sensing circuits comprising a first electrode sensing circuit and a second electrode sensing circuit, which are positioned parallel to each other and are in contact with the absorbent material, wherein the pair of electrode sensing circuits is laid out in a spatially distributed pattern across the absorption material; and
a controller configured to apply a voltage across the pair of electrode sensing circuits and to produce an electrical current that increases with area of the soiled area according to the spatially distributed pattern.

2. The smart diaper of claim 1, wherein the pair of electrode sensing circuits includes multiple curved portions, wherein the distance between adjacent curved portions is at least 5 times larger than a gap between the first electrode sensing circuit and the second electrode sensing circuit.

3. The smart diaper of claim 2, wherein the distance between adjacent curved portions is at least 10 times larger than a gap between the first electrode sensing circuit and the second electrode sensing circuit.

4. The smart diaper of claim 1, wherein the pair of electrode sensing circuits includes multiple straight portions, wherein the distance between adjacent straight portions is at least 5 times larger than a gap between the first electrode sensing circuit and the second electrode sensing circuit.

5. The smart diaper of claim 4, wherein the distance between adjacent straight portions is at least 10 times larger than a gap between the first electrode sensing circuit and the second electrode sensing circuit.

6. The smart diaper of claim 1, wherein the pair of electrode sensing circuits is laid out in a serpentine shape, a circular shape, a spiral shape, or an oval shape.

7. The smart diaper of claim 1, wherein the controller is configured to produce a current that is substantially proportional to the area of the soiled area according to the spatially distributed pattern.

8. The smart diaper of claim 1, wherein the absorption material forms an absorption layer, wherein the first electrode sensing circuit is positioned above the second electrode sensing circuit relative to the absorption layer.

9. The smart diaper of claim 1, wherein the absorption material forms an absorption layer, wherein the first electrode sensing circuit and the second electrode sensing circuit relative to the absorption layer are positioned side by side relative to the absorption layer.

10. The smart diaper of claim 1, wherein the pair of electrode sensing circuits is embedded in the absorbent material.

11. The smart diaper of claim 1, wherein the controller comprises a semiconductor chip in connection with the pair of electrode sensing circuits and configured to produce the voltage and to measure the electrical current that increases with the area of the soiled area.

12. The smart diaper of claim 11, wherein the controller comprises an antenna configured to transmit a wireless signal to alert a caregiver based on the electrical current measured by the semiconductor chip.

13. The smart diaper of claim 12, wherein the wireless signal alerts the caregiver when the electrical current measured the controller passes a threshold value.

14. The smart diaper of claim 12, wherein the wireless signal indicates area of the soiled area.

15. A smart diaper to be worn by a user, comprising:
a segmented measurement strip comprising:
a first electrode sensing circuit and a second electrode sensing circuit, which are positioned parallel to each other,
a plurality segments of absorbent material between the first electrode sensing circuit and the second electrode sensing circuit, wherein the absorption material is configured to absorb a liquid; and
segments of non-absorbent material between the plurality segments of absorbent material; and
a controller configured to apply a voltage across the first electrode sensing circuit and the second electrode sensing circuit and to produce an electrical current that increases with number of segments of absorbent material that is soiled by the liquid.

16. The smart diaper of claim 15, further comprising:
an absorption layer configured to absorb the liquid to form a soiled area, wherein the absorption layer is in contact with the plurality segments of absorption material.

17. The smart diaper of claim 15, wherein the non-absorbent material comprises a hydrophobic film to enhance liquid resistance.

18. The smart diaper of claim 15, wherein the first electrode sensing circuit and the second electrode sensing circuit includes a conductive ink placed on a layer of the absorbent material, wherein the segments of non-absorbent material comprises a hydrophobic film coated on the layer of absorbent material.

19. The smart diaper of claim 15, wherein the controller comprises a semiconductor chip in connection with the pair of electrode sensing circuits and configured to produce the voltage and to measure the electrical current that increases with the area of the soiled area.

20. The smart diaper of claim 19, wherein the controller comprises an antenna configured to transmit a wireless signal to alert a caregiver based on the electrical current measured by the semiconductor chip.
